(19) United States
(12) Patent Application Publication
(10) Pub. No.: US 2010/0152744 A1
(43) Pub. Date: Jun. 17, 2010

(54) Cement Applicator

(76) Inventor: Rudi Georg Bitsch, Heidelberg (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/516,291

(22) PCT Filed: Jan. 23, 2008
(86) PCT No.: PCT/EP08/50777

§ 371 Date: Feb. 18, 2010

(30) Foreign Application Priority Data
Feb. 1, 2007 (DE) 10 2007 005 025.0
May 22, 2007 (DE) 10 2007 024 083,1

(51) Int. Cl. A61B 17/56

(52) U.S. Cl. 606/94

(57) ABSTRACT

Process and device for shaping a moldable compound applied to a bone, in particular to a femur head, which sheathes an understructure for a head part of a prosthesis, in particular for a head part of a femoral implant, whereby that of the moldable compound is covered by a mold cap 8 with moldable inner space 11, whereby a guide element 2 is placed on the bone, which makes possible a smooth guiding of the mold cap 8, whereby the moldable compound is applied to the bone surface, and whereby the moldable compound is applied in a shaping way with the mold cap 8 that is guided by the guide element 2.

This invention relates to a process for shaping a moldable compound applied to a bone, in particular a femur head, in particular a bone cement for anchoring a prosthesis component, in particular a hip-joint surface replacement prosthesis, whereby a mold cap with a molding undersurface, in particular with a molding inner space, is pushed onto the moldable compound. Moreover, the invention relates to a device for implementing the process, whereby the device comprises a mold cap and a guide element.

In general, sustainment of the anatomy and biomechanics is a fundamental goal of the prosthetics, in particular also the hip endoprosthetics. In this case, it is important specifically in the care of young patients with hip joint replacement to maintain bone and to be minimally invasive as much as possible. Finally, by sustaining the bone, the anchoring quality in a later replacement operation that is not to be ruled out is improved. Manufacturers are increasingly attempting to take this requirement into account by developing surface replacement prostheses for the treatment of coxarthritis.

Surface replacement offers a treatment that is especially gentle on the bone. In this treatment, cartilage is removed from the hip bone and correctly shaped, and a hip cap is attached to the latter. The femur neck and the hip head remain. The hip socket is conventionally placed “press fit” into the pelvis. A special advantage of this surface replacement system is that in a possible later replacement operation, a “conventional” longitudinal shaft prosthesis can always still be implanted. Because of the reconstruction of the anatomical conditions, the cap system obtains the natural mobility and thus contributes to quick rehabilitation.

By now, surface replacement systems are implanted worldwide by many centers. In this case, the femoral attachment of the prosthesis is implemented mostly in cement form, whereby the primary attachment depends on the cementing technique. In this case, studies have shown that the rate of the aseptic loosening of the prostheses, which occurs between cement and bone and/or between cement and prosthesis, is higher than in the case of conventional longitudinal shaft prostheses and occurs on the femoral side in most cases. Thus, the cement-bone interface requires a sufficient penetration of the cement in the spongiosa on the proximal femur. Also, a wetting of this area of the bone or cement with blood can have disadvantageous effects on the two interfaces.

Moreover, an especially deep cement penetration or an especially thick cement layer can result in heat necroses in the bone. Thus, in human femora, temperatures of more than 50° C. were measured at a distance of 3 mm from the cement surface, which lasted longer than 30 seconds and thus could damage the bone tissue irreversibly. If, however, too much cement remains between bone and prosthesis, a poor prosthesis seat results therefrom, which is accompanied by biomechanically unfavorable lever ratios and an increased risk of femur neck fractures. Too much cement under a surface replacement prosthesis, in the bone or on the bone surface can cause bone necroses, the loosening of prostheses, and femur neck fractures.

To simplify the cementing, auxiliary tools and applicators were developed. The devices known to date replace the operator but are not able to distribute the cement in a controlled, uniform and smooth manner on the bone in order to produce a defined cement penetration in the bones. The known devices have the following drawbacks: on the one hand, the central opening provided for the guide pin of the prosthesis can close up, and thus air pockets can develop under the prosthesis cap. On the other hand, too little cement on the outer lower edge or too much cement at the prosthesis pole under the prosthesis cap can result in an incomplete prosthesis seat. Moreover, blood lamination and contamination by hand contact are known to be additional problems.

The object of the invention is now to propose a process that is simple to implement in practice, and a correspondingly comfortable-to-handle device for shaping the moldable compound that is applied to the bones, in particular to the femur head, which correct the above-mentioned drawbacks.

This object is achieved by a process with the features of claim 1 and a device with the features of claim 7. The features of the respective subclaims pertain to especially advantageous configurations of the invention.

The essential idea of the invention is to provide a guide element for the mold cap, which is placed on the bone and which makes it possible to smoothly guide the mold cap when it is pushed onto the moldable compound, in particular the bone cement. With this guide element, the mold cap can be moved in a defined way in terms of its direction and its pressing force, and the moldable compound can be formed. To this end, the moldable compound (“bone cement” below) is applied to the bone surface and pushed by the mold cap that is guided by the guide element while being shaped. In the device according to the invention, the guide element thus forms a guide for the mold cap, whereby the mold cap can move axially on the guide element.

With the use of the invention, the bone cement can be polished and matched to the later prosthesis form. Moreover, a defined pressure gradient, which promotes the penetration of the bone cement into the bones, can be built up. Moreover, the pressure gradient can be matched to the viscosity of the moldable compound, and a controlled penetration of the moldable compound in the bones can be ensured. When attaching the prosthesis, the bone cement connects the sponge-like bone surface to the anchoring elements of the prosthesis undersurface and hardens. Excess cement can be removed in a simple way at the prosthesis pole. An understructure that can support loads, which makes the incorporation of the bone into the undersurface of the prosthesis unnecessary and which results in a uniform application of force without stress peaks, is formed.

In this case, it is basically all the same whether first the guide element is placed and then the bone cement is applied or whether the bone cement that is first applied is later penetrated by the guide element. More advantageously, however, the guide element is first placed on the bone before the bone cement is applied to the bone surface. As a result, the central opening in the prepared bone is sealed before the bone cement is applied, so that its penetration of the cement into the opening is avoided. In this case, the advantage of the free central opening is that when the cup-shaped surface replacement prosthesis is placed, the escape of air out from under the latter can be ensured.

Accordingly, the basic idea is to guide the mold cap by means of the guide element securely into a position in the bone that corresponds to the later occupied position of the prosthesis component. In this case, the shaping of the bone cement can be quickly implemented by the smooth guiding of the mold cap. After the mold cap is removed, enough time remains for the removal of possibly projecting material and for pressing the prosthesis before the bone cement hardens. Studies have shown that a cement penetration depth of 2-3 mm is enough to achieve interlocking with a bone trabecula that runs transversally. For an optimum prosthesis anchoring, a cement penetration with a depth of between 2 and 5 mm is therefore recommended. Using the invention, these requirements can be achieved reliably and comfortably.

The process and the device are not limited to the application on a femur head or on the surface replacement prostheses of individual manufacturers, but rather it can primarily also be used for applications on other joints and for prostheses of other manufacturers.

In this case, after the preparation of the femur head, the purification and the drying, the inventive process comprises in particular the following process steps:

In a first process step, the guide element is placed on the bone so that it can design a smooth guide for the mold cap. In this case, it is advantageous if the guide element is designed as a metal pin that is inserted into a prepared opening in the bone. In the next process step, the bone cement is applied to the bone surface, and the latter is completely covered with the bone cement. The application can take place with a cement pistol. In this case, it is not necessary to touch or press the bone cement with the fingers. Subsequently, the mold cap that is smoothly guided by the guide element can be pressed uniformly on the bone cement, and the bone cement can be shaped into a homogeneous layer with as uniform a thickness as possible.

In an especially preferred embodiment, the guide element has axial sections, whereby a first axial section corresponds at least partially to a form of a centering pin that is present in the head part of the prosthesis. In this way, the guide element can be used and thus oriented in the central opening, provided for the centering pin, in the prepared bone. In this case, the guide element is optimally centered on the bone, whereby the handling is especially suitable for practice. Moreover, because of the at least partially consistent shapes, the dimensions of the central opening in the bone, i.e., the stability and the clearance of the opening for the centering pin of the prosthesis, can still be checked before the prosthesis is placed.

A second axial section of the guide element forms a sliding fit for a central hole in the cap bottom of the mold cap, by which the mold cap can be guided through the guide element in an especially simple way. The diameter of the hole is matched to the diameter of the guide element according to the requirements of the sliding fit.

In another preferred embodiment, the second axial section of the guide element has a larger diameter than the first axial section of the guide element, whereby the transition region forms a stage. This stage attaches at the edge of the bone surface when the guide element is inserted into the central opening. A certain clearance distance around the central opening in the bone is provided by the stage, which is not covered by the bone cement. As a result, it is ensured that when the prosthesis is applied, the trapped air can escape through the free central opening up to the complete prosthesis seat.

To make possible a comfortable handling by the operator, the mold cap is equipped in a preferred embodiment with a handhold that advantageously is connected in a detachable manner to the mold cap. The handhold is then fastened to the mold cap, before the latter is pressed by the guide element in a centered way to push the bone cement onto the bone surface while shaping it. By the connection with the handhold, the mold cap is especially easy to handle. The detachable attachment also contributes to a better purification of the individual parts. Preferably, the guide element and handhold form a display that indicates the distance of the molding inner space of the mold cap from the bone surface. If the display shows scaling, the operator is informed at all times about the remaining layer thickness as the bone cement is being pushed on. Preferably, a stop is provided, which defines an end position between the mold cap and the guide element and thus the mold cap relative to the prepared bone. The mold cap can be pressed by the operator on the molding compound until the stop is reached, whereby the stop is adjusted so that the desired distance between the mold cap and the surface of the bone is ensured.

In another especially preferred embodiment, during the pushing, air that is trapped under the mold cap and/or a portion of the moldable compound can escape through at least one outlet opening in the cap bottom. Pockets of the moldable compound at the prosthesis pole, an excessive pressure gradient, and thus too low a penetration of the moldable compounds are avoided because of these outlet openings. As a result, a pressure release and reduction of the amount of moldable compound in the area of the problem zone of the prosthesis pole can be carried out. The number and diameter of the outlet openings in the cap bottom are preferably to be construed according to the viscosity of the bone cement. For a bone cement of average viscosity, in particular three symmetrical outlet openings that are arranged around the central opening of the cap bottom with a respective diameter of about 4 mm are suitable.

The outer edge of the mold cap is preferably conically chamfered. This ensures that the bone cement that is displaced when the mold cap is applied is pressed onto the bone surface. Thus, a pressure gradient, which leads to an adequate penetration of the bone cement in the bone, is also produced on the outer edge of the mold cap.

The mold cap can be removed from the cement surface after reaching the desired distance with a rotational movement on the handhold. In the device according to the invention, no bone cement remains stuck on the mold cap, and a comparatively smoother jacket made of bone cement is produced, which has an especially uniform layer thickness and homogeneous density on the bone surface. After the removal of the mold cap, the bone cement left at the lower edge of the prosthesis bed can easily be removed while being watched visually. No particles of the bone cement are left over or remain near the later artificial joint.

The especially uniformly thick layer of the moldable compound on the bone is used to produce as secure a connection as possible with the femoral prosthesis components and is further compressed by the latter during settling. An incomplete prosthesis seat can thus be ruled out because of too great or too dissimilar a layer thickness of the moldable compound.

In addition, the process according to the invention has the advantage that during the application of the mold cap, the compressive force corresponds to the compressive force that has to be exerted in the further course of the operation on the prosthesis. The operator can detect how quickly the mold cap moves under its manual force and is set on the moldable compound. For the operator, it is thus simpler to select and to maintain the correct application of pressure on the prosthesis.

The cap material should be sufficiently solid not to become deformed during pressing. The mold caps are preferably made completely from Teflon to avoid adhesion of the moldable compound. For reasons of economy, the mold cap can also be produced from another material. The cap material should make it possible to produce smooth inner surfaces that optionally can be provided with an anti-adhesive or Teflon coating. To prevent scratching of the mold cap and thus adhesion of the moldable compound, the latter should be used only a few times or should be designed as a disposable product.

The guide element preferably consists of high-grade steel; also, another more advantageous material with sufficient strength could be used here.

With the process according to the invention and the device according to the invention, a more reliable connection of the prosthesis to the bone can be ensured by a homogeneous cement understructure. In this case, the process and the device are distinguished by high suitability for practical use. Also, the thorough purification and the disinfection of individual parts is easily possible, whereby it is advantageous, as stated above, to produce the device as a disposable item.

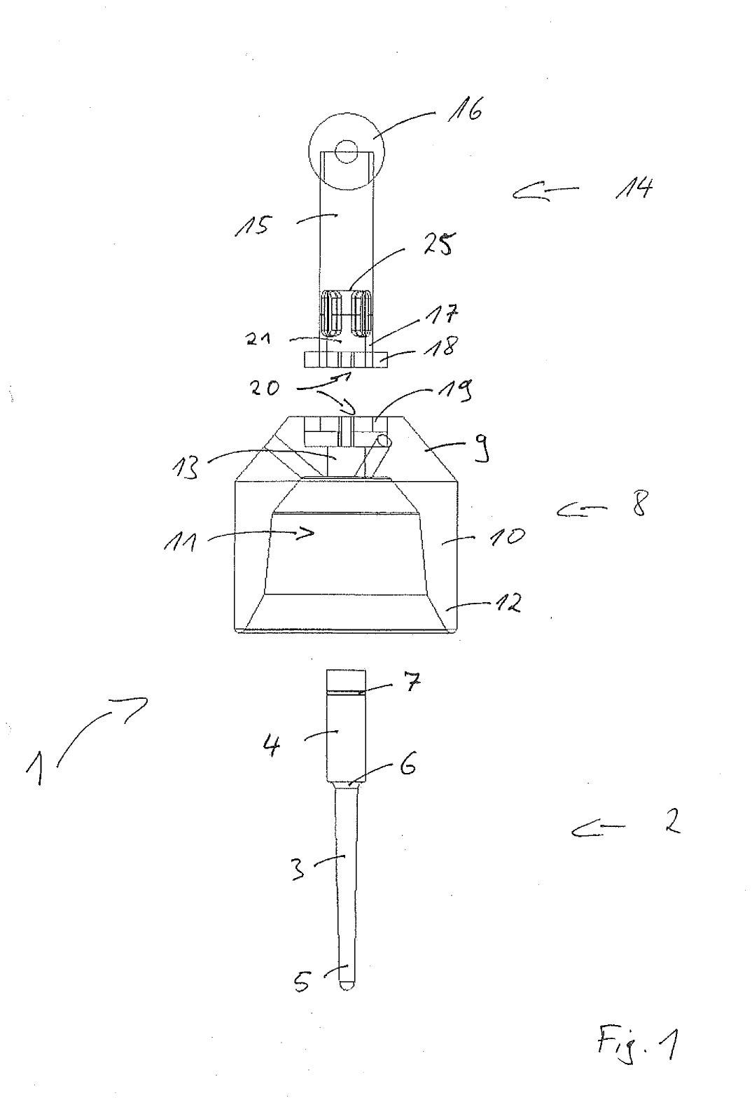

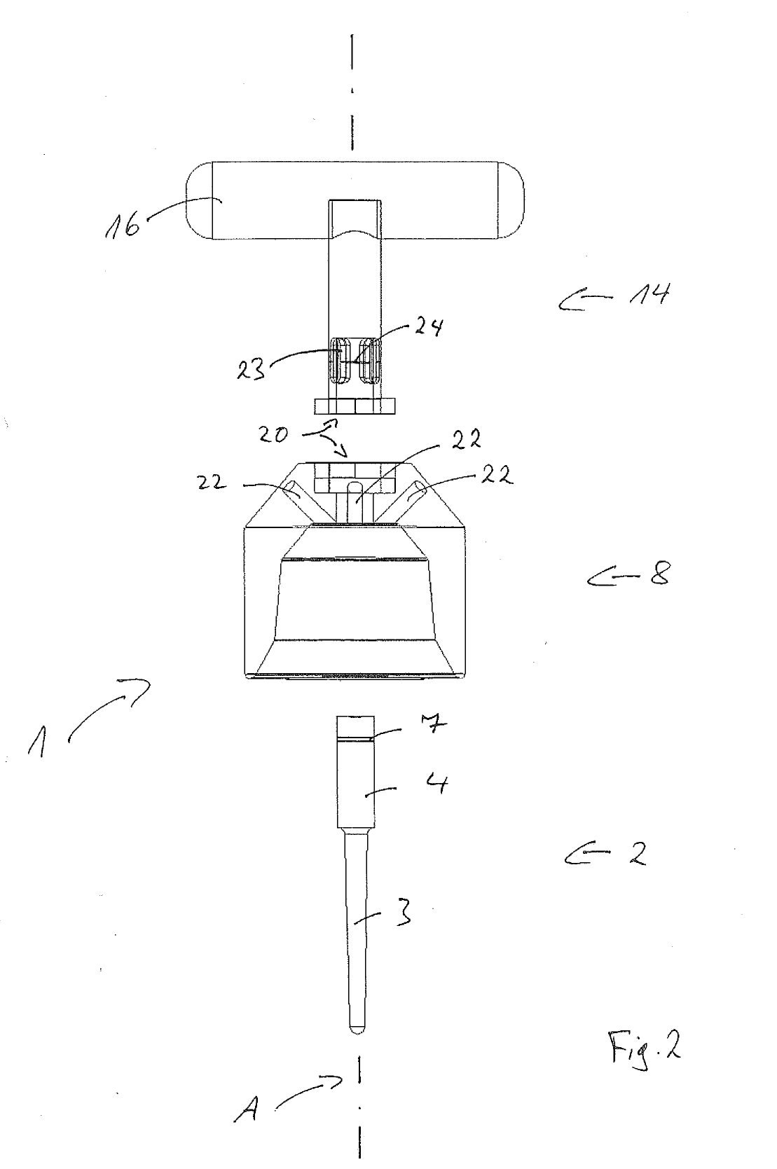

Below, the invention is explained in more detail based on FIGS. 1 and 2. In this case:

FIG. 1 shows a sectional view through an embodiment of the device according to the invention, and

FIG. 2 shows a sectional view of the device according to the invention that is rotated by 90°.

The device 1 that is shown in FIG. 1 consists of three components. The axisymmetrical guide element 2 as one of the components has two axial sections 3 and 4. The first axial section 3 of the guide element 2 with a tip 5 with a slightly conical taper corresponds in its geometry to a centering pin of a surface replacement prosthesis, for example the centering pin of an ASR (Articular Surface Replacement) surface replacement prosthesis for the femur head. By coordinating the sizes, the first axial section 3 can be completely inserted into a central opening that is provided for the centering pin in the prepared femur head.

The second axial section 4 of the guide element 2 has a cylindrical shape and has a larger diameter than the first axial section 3. The transition between the first section 3 and the second section 4 is formed as a stage 6 that is placed on the edge of the bone that surrounds the opening during insertion of the first axial section 3 into the central opening of the femur head. Moreover, the axial section 4 has one or more markings 7 that run in a circle around the cylindrical surface in the peripheral direction, and said surface is part of a display for determining the penetration depth.

Another component of the device 1 is the mold cap 8. The mold cap 8 has a cap bottom 9 of a specific thickness and an outside wall 10, whereby cap bottom 9 and wall 10 have a molding undersurface, which is designed as an inner space 11 with a smooth surface. The molding undersurface 11 of the mold cap 8 has—relative to its inside angle—an identical inner geometry, but larger in its diameter than the undersurface of the prosthesis that is to be placed. This has the purpose that during application of the prosthesis, the bone cement still has the potential, to some extent, to be compressed, which ensures a more reliable connection of the prosthesis undersurface and cement. For a prosthesis of the size 49, a cement jacket thickness of 1 mm is to be preferred, and the diameter of the inner geometry of the molding inner space 11 is to operate about 2 mm larger than the inner space of the prosthesis. The mold cap 8 is made of Teflon, whereby in principle, it is enough to line the molding undersurface with Teflon or another anti-adhesive coating.

At its edge 12, the mold cap 8 is “chamfered out” and runs out conically at an angle of about 30°. The edge 12 thus opens outwards and has an increasing inside diameter in comparison to the outer wall 10.

In the cap bottom 9, a central hole 13 is provided, whose diameter is designed for a smooth guiding of the cylindrical, second axial section 4 of the guide element 2.

A handhold 14 as a third component of the device 1 has a cylindrical neck 15 and a handle 16, which are connected securely to one another. At the end 17 of the handle neck 15, two facing radial attachments 18, together with radial recesses 19 arranged outside of the central hole 13 of the cap bottom 9, form a bayonet closure 20, by means of which the handhold 14 and the mold cap 8 can be connected in a detachable manner. A hole 21, in which the second axial section 4 of the guide element 2 can be inserted up to a stop 25 in the application of pressure of the mold cap 8, can be inserted into the neck 15.

FIG. 2 shows a section rotated by 90° through the device according to the invention corresponding to FIG. 1. To apply a cement compound applied to the prepared femur head in a shaping way, the three components 1, 8 and 14 of the device 1 are oriented axially along a common center axis A. The handhold 14 that is designed in the shape of a T is connected for this purpose via the bayonet closure 20 by a 90° rotation with the mold cap 8. Then, the mold cap 8 is applied on the guide element 2, and the second axial section 3 is inserted into the central hole 13 of the mold cap 8.

The guide element 1 that is introduced with its first axial section 2 into the central opening of the prepared femur head runs the mold cap 8 over the second axial section 3 that slides into its central hole 13, so that the mold cap 8 is centered over the central hole 13. If pressure is now exerted on the mold cap 8 via the handhold 14 along the center axis A, the cement compound is applied uniformly in a shaping manner by the mold cap 8 that is guided by the second axial section 4 and thus is centered on the femur head.

Air and/or excess cement can escape during the application on the bone surface through three outlet openings 22 that are arranged symmetrically in the cap bottom 9, radically outside of the central hole 13. Slots 23 in the handle neck 15 make it possible for the operator to see the marking 7 of the axial section 4 of the guide element 2 accommodated in the inner hole 21. With the marking 7, scale 24 on the outer periphery of the handle neck 15 forms a display on which the remaining thickness of the cement compound can be read while it is being applied.

The inner hole 21 forms the stop 25 for the second axial section 4. The stop 25 determines an end position of the mold cap 8 on the cement compound, which is designed according to the desired thickness of the cement compound. For a preferred layer thickness of the formed cement compound of 1 mm, the stop 25 for an end position of the molding inner space 11 of the mold cap 8 is to be selected 1 mm above the prepared femur head.

Via this device, it is possible that the compressive force during molding application by means of the mold cap 8 corresponds at least approximately to the compressive force that is exerted when the prosthesis is set.

1. Process for shaping a moldable compound applied to a bone, in particular a
femur head, in particular bone cement, for anchoring a prosthesis component, in particular a hip-joint surface replacement prosthesis, whereby a mold cap (8) with a molding undersurface, in particular with a molding inner space (11), is pushed onto the moldable compound,
characterized in that
a guide element (2) is placed on the bone, which makes possible a smooth guiding of the mold cap (8),
in that the moldable compound is applied to the bone surface, and
in that the moldable compound is applied in a shaping way with the mold cap (8) that is guided by the guide element (2).

2. Process according to claim 1,
wherein
first, the guide element (2) is applied on the bone, before the moldable compound is applied to the bone surface.

3. Process according to claim 1,
wherein
the guide element (2) is inserted in an opening that is present in the bone until stage (6) that is present in the guide element (2) is applied to the bone surface.

4. Process according to claim 3,
wherein
with the placement of the guide element (2), the dimensions of the central opening of the bone provided for a centering pin of the prosthesis are checked.

5. Process according to claim 1,
wherein
the mold cap (8) has a handhold (14), whereby the mold cap (8) is pressed by means of the handhold on the moldable compound until the guide element (2) strikes against a stop (22) that is provided in the handhold.

6. Process according to claim 1,
wherein
the surface of the moldable compound is polished by the application by the mold cap (8) and in particular occupies a uniform thickness on the bone.

7. Device for shaping a moldable compound to be applied to a bone, said device comprising a mold cap (8) and a guide element (2),
wherein
the guide element (2) forms a guide for the mold cap (8), whereby the mold cap (8) can move axially on the guide element (2).

8. Device according to claim 7,
wherein
the pin-shaped guide element (2) that is made in particular of metal has sections (3, 4) of different diameters, whereby a first section (3) corresponds at least partially to the form of the centering pin that is present in the head part of the prosthesis, and whereby a second section (4) forms a sliding fit for the mold cap (8) that is equipped with a central hole (13).

9. Device according to claim 8,
wherein
the second section (4) has a larger diameter than the first section (3), whereby the transition zone between the sections forms the stage (6).

10. Device according to claim 7,
wherein
the molding inner space (11) of the mold cap (8) is smooth and corresponds to the geometry of the surface of the prosthesis, whereby it is larger in particular by a pressing distance.

11. Device according to claim 7,
wherein
the mold cap (8) has an edge (12) that opens conically.

12. Device according to claim 7,
wherein
the cap bottom (9) has at least one outlet opening (22) for the escape of excess moldable compound and/or air.

13. Device according to claim 7,
wherein
the mold cap (8) is made of Teflon or the molding inner space is lined with Teflon or another anti-adhesive coating.

14. Device according to claim 7,
characterized by
a handhold (14) that can be fastened in particular in a detachable manner to the mold cap (8) with an axial hole (21), which occupies the second axial section (4) of the guide element (2) during molding application.

15. Device according to claim 14,
wherein
a stop (25), which defines an end position for the molding, is provided in the hole (21).

16. Device according to claim 7,
wherein
the handhold (14) and guide element (2) form a display for determining the penetration depth.
